(19) United States
(12) Patent Application Publication
(10) Pub. No.: US 2024/0225460 A1
(43) Pub. Date: Jul. 11, 2024

(54) METHODS FOR QUANTIFYING BLOOD PRESSURE STABILITY

(71) Applicant: UNIVERSITY OF LOUISVILLE RESEARCH FOUNDATION, INC., Louisville, KY (US)

(72) Inventors: Susan J. Harkema, Louisville, KY (US); Beatrice Ugiliweneza, Louisville, KY (US); Samineh Mesbah, Louisville, KY (US)

(21) Appl. No.: 18/555,308

(22) PCT Filed: Apr. 15, 2022
(86) PCT No.: PCT/US2022/024981

§ 371 Date: Oct. 13, 2023

Related U.S. Application Data
(60) Provisional application No. 63/175,371, filed on Apr. 15, 2021.

(51) Int. Cl. A61B 5/022

(52) U.S. Cl. CPC A61B 5/022

(57) ABSTRACT

A novel method for assessing blood pressure stability uses a plurality of outcome parameters based on blood pressure measurements. An individual subject may be classified as of hypotensive, hypertensive, unstable, or normal based on the plurality of outcome measures, which are calculated based on a target blood pressure range and a plurality of expanding blood pressure ranges.

### CROSS-REFERENCE TO RELATED APPLICATION

This Application claims the benefit of U.S. provisional patent application Ser. No. 63/175,371, filed Apr. 15, 2022, for METHODS FOR QUANTIFYING BLOOD PRESSURE STABILITY, incorporated herein by reference.

### FIELD OF THE INVENTION

A novel method for assessing blood pressure stability uses a plurality of outcome parameters based on blood pressure measurements. An individual subject may be classified as of hypotensive, hypotensive, unstable, or normal based on the plurality of outcome measures, which are calculated based on a target blood pressure range and a plurality of expanding blood pressure ranges.

### BACKGROUND OF THE INVENTION

Spinal cord injury (SCI) leads to life-long autonomic cardiovascular dysfunction for which there is no adequate treatment. For the estimated millions of people living with SCI globally, altered autonomic cardiovascular (CV) regulation leads to chronic arterial blood pressure instability with variation between hypotension that can be exacerbated by orthostasis and severe hypertension triggered by autonomic dysreflexia. Studies on individuals with chronic SCI have identified blood pressure instability as one of the determinants of cardiovascular morbidity and mortality, including a 4-fold increased stroke risk in the SCI population. Even in those without SCI, mounting evidence links blood pressure instability during orthostasis with poorer general physical and mental health.

Recent studies have shown that spinal cord epidural stimulation (scES) is able to stabilize blood pressure toward a more normotensive range and alleviation of symptoms of orthostatic hypotension when using cardiovascular-specific stimulation parameters. It is suggested that scES may facilitate adaptive neuroplasticity to restore the underlying autonomic nervous system defect, leading to improved CV regulation after severe chronic SCI. It has been shown that the profound variability between blood pressure measurements observed in individuals with SCI does not occur in individuals receiving scES optimized for cardiovascular function, and it is not seen in non-injured individuals. However, outcome measures of cardiovascular dysfunction that are both statistically and clinically relevant and can detect change in blood pressure and identify a relationship to a normative range are limited and there is no comprehensive measure designed to accurately quantify the complex dynamic changes that occur during continuous blood pressure recordings in individuals experiencing cardiovascular dysfunction.

### SUMMARY

To address the identified challenges, Applicant presents a novel method for assessing blood pressure based on the cumulative distribution of data points within and outside of a normative range. This straightforward and intuitive method can comprehensively capture the complex and dynamic blood pressure variability that SCI population experience in their day-to-day life. The method provides a reliable means to accurately quantify effects of SCI on blood pressure instability, as well as provide a foundation for statistical comparison among SCI groups, e.g., in response to perturbation, or with and without interventions to stabilize blood pressure.

In some embodiments, the present invention is a method for assessing blood pressure stability in a living subject, the method comprising the steps of: measuring a blood pressure of a living subject over a period of time, said measuring including collecting a plurality of data points; defining a target blood pressure range, said range including a blood pressure target value centered within the target blood pressure range; calculating a total deviation of data points from the blood pressure target value; defining a plurality of expanded blood pressure ranges; plotting a cumulative distribution curve based on the data points and the plurality of expanded blood pressure ranges; fitting an exponential cumulative function to the cumulative distribution curve; determining a plurality of outcome measures, the plurality of outcome measures including the total deviation and at least one of an x-intercept of the exponential cumulative function, a y-intercept of the exponential cumulative function, λ, In(λ), and a fitting error calculated from said fitting; and assessing blood pressure stability of the living subject based on the plurality of outcome measures. In certain embodiments, the measured blood pressure is a systolic blood pressure, wherein the blood pressure target range is a systolic blood pressure target range, wherein the blood pressure target value is a systolic blood pressure target value; and wherein the plurality of data points are a plurality of systolic blood pressure values collected over time. In further embodiments, the target blood pressure range is 110 mmHg to 120 mmHg, and wherein the blood pressure target value is 115 mmHg. In some embodiments, prior to calculating the total deviation of data points, a first portion of data points above the target blood pressure range are omitted and a second portion of data points below the target blood pressure range are omitted. In further embodiments, the first portion of data points is the highest data points above the target blood pressure range, and wherein the second portion of data points is the lowest data points below the target blood pressure range. In certain embodiments, the first portion of data points is 5% of the total data points and the second portion of data points is 5% of the total data points. In some embodiments, prior to said calculating a total deviation of data points, the data points are downsampled. In further embodiments, an area under the curve (AUC) is calculated for the cumulative distribution curve, and said plurality of outcome measures include the AUC. In certain embodiments, the exponential cumulative function is fitted to the cumulative distribution curve using the equation

$$Y = \{ \begin{matrix}( 1 - {e}^{- λ ( X - {X}_{0} )} ) + {Y}_{0} & X \geq 0 \\ 0 & X < 0\end{matrix} .$$

In some embodiments, the living subject is classified as one of as one of hypotensive, hypertensive, unstable, or normal based on the assessment of blood pressure stability. In other embodiments, the living subject is classified as having stable or unstable blood pressure based on the assessment of blood pressure stability. In certain embodiments, the plurality of expanded blood pressure ranges vary by a predetermined expansion rate. In some embodiments, the expansion rate is between 1 mmHg and 20 mmHg. In certain embodiments, the expansion rate is between 1 mmHg and 10 mmHg. In further embodiments, the expansion rate is not more than 10 mmHg. In some embodiments, the method is performed by a computing system using at least one processor receiving the plurality of data points as input. In certain embodiments, the plurality of outcome measures includes at least one of, at least two of, at least three of, at least four of, at least five of, or at least six of the total deviation, the AUC, an x-intercept of the exponential cumulative function, a y-intercept of the exponential cumulative function, λ, In(λ), and a fitting error calculated from said fitting.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1A is a depiction of an apparatus for measurement of finger blood pressure (beat-to-beat) and brachial blood pressure (used for calibration) recording.

FIG. 1B is a chart depicting systolic (upper line) and diastolic (lower line) blood pressure recordings (calibrated beat-to-beat data, downsampled by 50 points) in supine and sitting positions during sit-up test from an individual with chronic SCI.

FIG. 1C is a chart depicting systolic and diastolic blood pressure recordings (calibrated beat-to-beat data, downsampled by 50 points) in supine and 70° tilt positions during 70° tilt maneuver from an individual with chronic SCI.

FIG. 2A is a pictorial representation of brachial blood pressure recording and a table of recorded systolic blood pressure (SBP) measurements over 6 hours of awake time in an individual with chronic SCI.

FIG. 2B is a plot depicting six hours of systolic blood pressure measurements during awake time. The shaded area illustrates the 110-120 mmHg target range, while the horizontal dashed line extending through the center of the shaded area indicates the center of the blood pressure target range (i.e., 115 mmHg).

FIG. 2C is the plot of FIG. 2B with horizontal dashed lines to indicate the 5th (lower limit) and 95th (upper limit) percentiles of SBP measurements. Two vertical curly brackets indicate the distance between the upper and lower limits and the 115 mmHg target value (dashed line), which provides the deviation above target value and deviation below target value. The sum of these two deviations provide the total deviation from the 115 mmHg target value.

FIG. 2D is the plot of FIGS. 2B and 2C with Y-axis limits expanded to include 40-230 mmHg. Horizontal gradient dashed lines mark each expansion of the upper and lower boundaries beyond the target range (expansion rate is set at 10 mmHg); the adjacent table shows the percentage of data points within each expanded range.

FIG. 2E is a plot depicting the cumulative distribution curve: the percentages of systolic blood pressure measurements are plotted against their corresponding ranges. The adjacent table shows the steps to calculate the area under the curve

(AUC) from summation of trapezoidal areas between the curve and X-axis.

FIG. 2F is a plot fitting the exponential cumulative function (shown in solid line) to the cumulative distribution curve (shown in dashed line): A is the rate parameter that shows the slope, X₀ and Y₀ show the intercepts with X and Y-axis, and the fitting error (E) is the difference between the cumulative curve and the exponential curve.

FIG. 2G is a table displaying the six outcome measures: total deviation from target, AUC, In(λ), X₀, Y₀, and E.

FIG. 3A (Top panel) a chart depicting systolic blood pressure measurements with hypotensive pattern recorded from a non-injured individual with data points distributed within or close to the normative 110-120 mmHg range (shaded area). The vertical curly brackets show the deviation of data points above and below 115 mmHg. Measurements are recorded using finger beat-to-beat blood pressure recording during 15 minutes in a sitting position and the data points are downsampled by 50 points. (Bottom panel) a chart depicting corresponding cumulative distribution curve (in data points and dotted line) and fitted exponential curve (in solid line) and the values of 6 outcome measures reported in text.

FIG. 3B (Top panel) a chart depicting systolic blood pressure measurements with hypotensive pattern recorded from an individual with SCI with data points distributed below the normative 110-120 mmHg range (shaded area). The vertical curly bracket shows the deviation below 115 mmHg. As in FIG. 3A, measurements are recorded using finger beat-to-beat blood pressure recording during 15 minutes in a sitting position and the data points are downsampled by 50 points. (Bottom panel) a chart depicting corresponding cumulative distribution curve (in data points and dotted line) and fitted exponential curve (in solid line) and the values of 6 outcome measures reported in text.

FIG. 3C (Top panel) a chart depicting systolic blood pressure measurements with an unstable distribution pattern recorded from an individual with SCI with data points distributed above and below the normative 110-120 mmHg range (shaded area). The vertical curly brackets show the deviation above and below 115 mmHg. As in FIGS. 3A and 3B, measurements are recorded using finger beat-to-beat blood pressure recording during 15 minutes in a sitting position and the data points are downsampled by 50 points. (Bottom panel) a chart depicting corresponding cumulative distribution curve (in data points and dotted line) and fitted exponential curve (in solid line) and the values of 6 outcome measures reported in text.

FIG. 4 is a series of box plots and individual values of stability outcome measures for SBP recorded from non-injured (NI) and SCI groups during sit-up test (during sitting position), 70° tilt maneuver (during the tilt) and 24-hour blood pressure monitoring (the awake period) for (panel A) total deviation from 115 mmHg; (panel B) area under the cumulative distribution curve (AUC); (panel C) integrated value for logarithm of rate parameter and x- and y-axis intercepts of fitted exponential function; and (panel D) fitting error (E). ES: Effect Size.

FIG. 5 is a series of average plots and individual values of stability outcome measures for SBP recorded from NI and SCI groups during sit-up test (supine versus sitting in SCI) and 70° tilt maneuver (supine versus tilt in SCI) for (panel A) total deviation from 115 mmHg; (panel B) area under the cumulative distribution curve (AUC); (panel C) integrated value for logarithm of rate parameter and x- and y-axis intercepts of fitted exponential function; and (panel D) fitting error (E).

FIG. 6 is a series of average plots and individual values of stability outcome measures for systolic blood pressure recorded from NI and SCI groups during sit-up test (15 minutes supine, day 1 versus day 2), 70o tilt maneuver (5 minutes supine, day 1 versus day 2) and 24-hour blood pressure monitoring (awake period, day 1 versus day 2) for (panel A) total deviation from 115 mmHg; (panel B) area under the cumulative distribution curve (AUC); (panel C) integrated value for logarithm of rate parameter and x- and y-axis intercepts of fitted exponential function; and (panel D) fitting error (E). ICC: Intra-class correlation.

FIG. 7 is a series of box plots and individual values of stability outcome measures for systolic blood pressure data recorded from SCI groups with and without scES intervention for cardiovascular regulation for (top left panel) total deviation from 115 mmHg; (bottom left panel) area under the cumulative distribution curve (AUC); (top right panel) integrated value for logarithm of rate parameter and x- and y-axis intercepts of fitted exponential function; and (bottom left panel) fitting error.

### DETAILED DESCRIPTION OF THE PREFERRED EMBODIMENTS

Systolic blood pressure data from individuals with SCI as well as non-injured participants was used to evaluate stability of blood pressure in response to perturbation and over a prolonged period of time.

### Cardiovascular Perturbation Tests

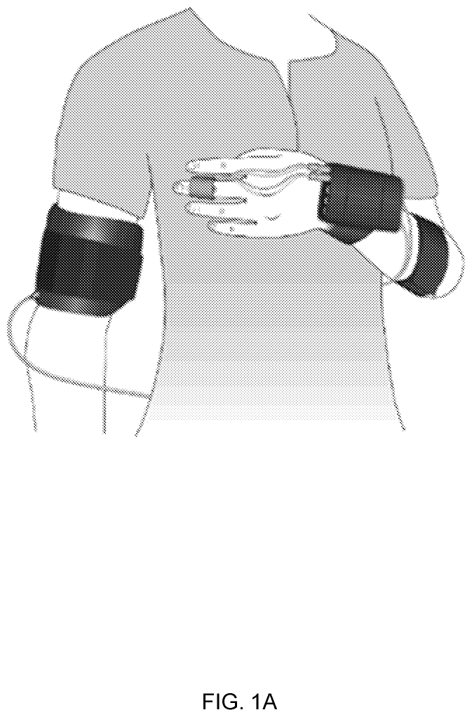

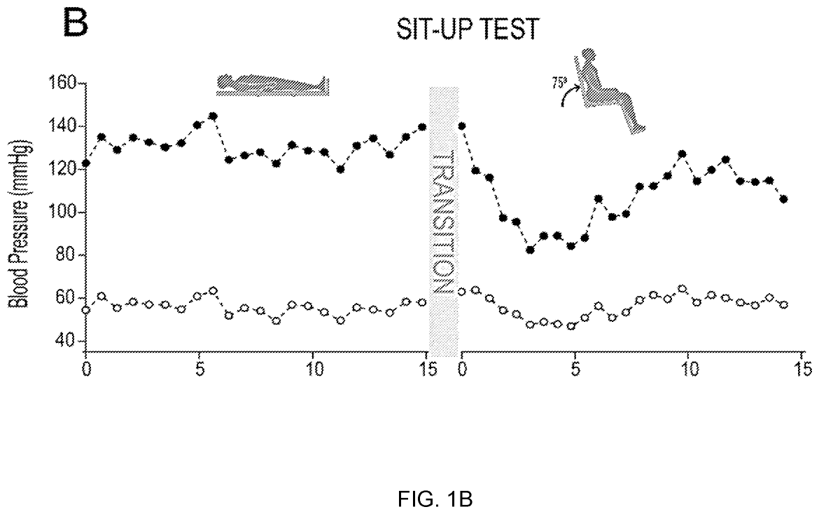

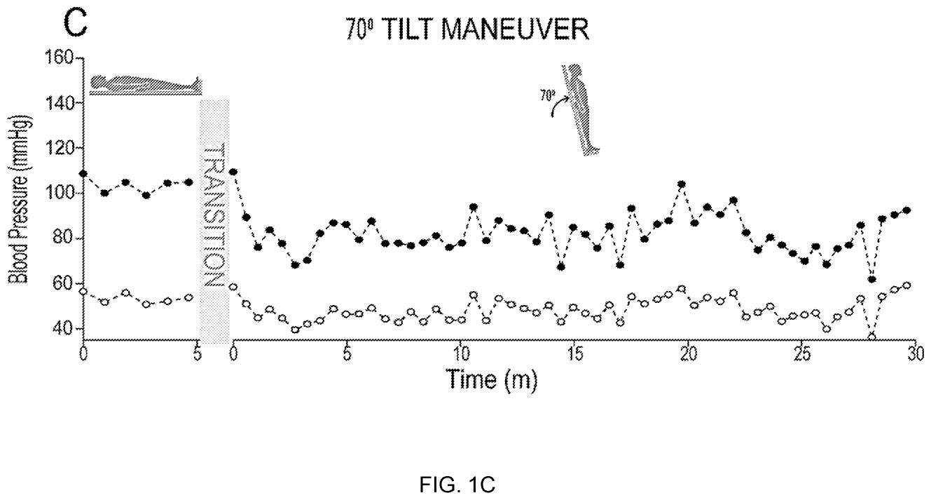

Sit-up test: Individuals lay supine for 5 minutes or 15 minutes and then were passively moved to the seated position with hips and knees at 90° angle (FIGS. 1A and 1B). The participants remained in seated position for 5 minutes or 15 minutes or as long as they could tolerate without showing symptoms of presyncope. In total, blood pressure data from 45 individuals with SCI and 48 non-injured participants (with no known cardiovascular dysfunction) were used for this test.

70° tilt test: Individuals lay supine for 5 minutes and once supported by restraints at the knees, hips, and chest the table would tilt upright to a 70° angle (FIGS. 1A and 1C); individuals would remain upright for 30 minutes or as long as tolerated without experiencing symptoms of presyncope. Blood pressure data of 34 individuals with SCI and 9 non-injured participants (with no known cardiovascular dysfunction) were used for this test.

In both of the aforementioned perturbation assessments, beat-by-beat systolic and diastolic blood pressure were obtained from finger plethysmography with intermittent brachial blood pressure measurements. Finger blood pressure measurements were calibrated to brachial blood pressure measurements offline using a 2-point calibration method.

### Twenty Four-Hour Blood Pressure Monitoring

The outcome measures of the disclosed toolset were also tested with systolic blood pressure data measured over 24 hours to ensure the toolset could evaluate what individuals experience daily. Blood pressure of individuals with SCI and Non-injured individuals was monitored over 24-hours using an automatic brachial recording device. Blood pressure data was recorded every 15 minutes during awake time and every 30 minutes during overnight sleeping; the schedule was determined in advance by each individual. All participants kept a diary of their daily routines including the time they slept at night and the time they woke up in the morning. Participants with too many missing data points were excluded. In total, the data from 22 individuals with SCI and 12 Non-injured participants (i.e., individuals with no known cardiovascular dysfunction) were used for the analysis.

### Six-Hour Blood Pressure Recording With and Without scES Intervention

Blood pressure data were recorded every 10 or 15 minutes over 6 hours of awake time using an automatic brachial recording device from two groups of individuals with SCI: one group with implanted scES targeting cardiovascular regulation (n=9) and a control group without scES (n=15). The blood pressure stability outcome measures developed herein were used to confirm whether these measures can accurately describe the cardiovascular effects of scES on blood pressure stability in individuals with SCI.

### Development of the Blood Pressure Stability Methodology

This method disclosed herein describes the distribution of SBP data with respect to the 110-120 mmHg target range. The selection of this range as the target does not imply that all individuals should have a SBP within 110-120 mmHg. Rather, it is a physiologically meaningful target range because the participants' pre-injury SBP is unknown (i.e., their own “healthy” systolic blood pressure) and 110-120 mmHg is within the “healthy” systolic blood pressure range established by the American College of Cardiology. While the methodology is described with respect to a target range of 110-120 mmHg, in other embodiments, other target ranges may be used, such as, for example, 105-125 mmHg, 100-129 mmHg, 105-115 mmHg, 115-125 mmHg, or 90 - 129 mmHg.

### Total Deviation from Target

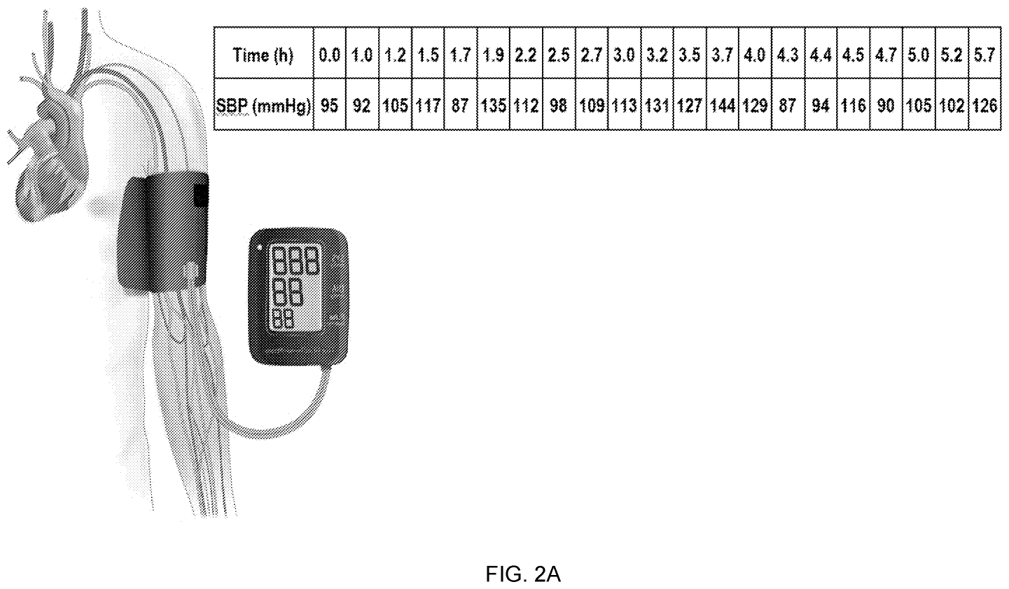

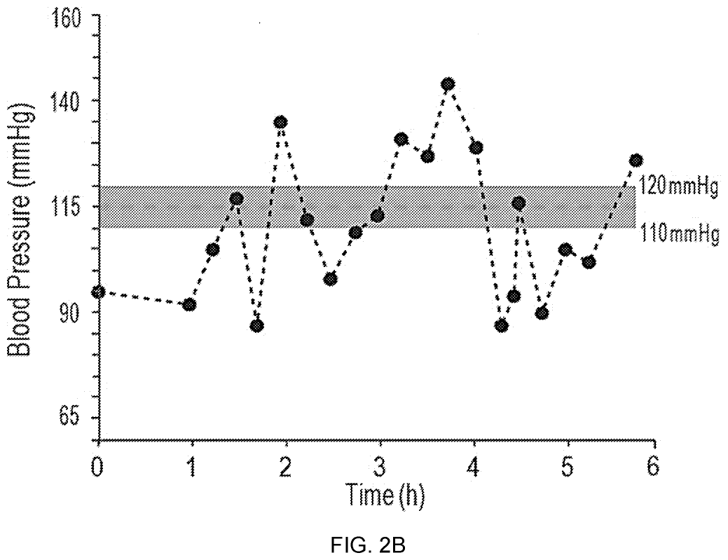

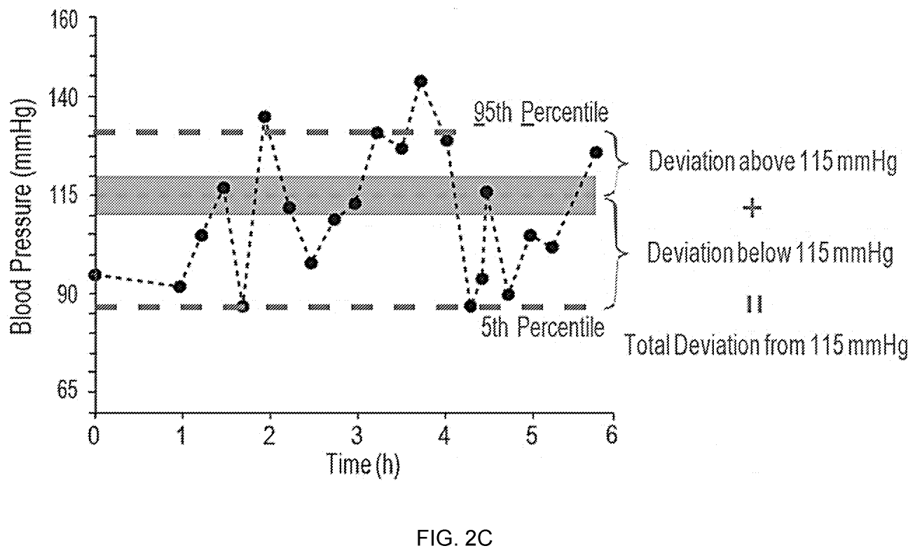

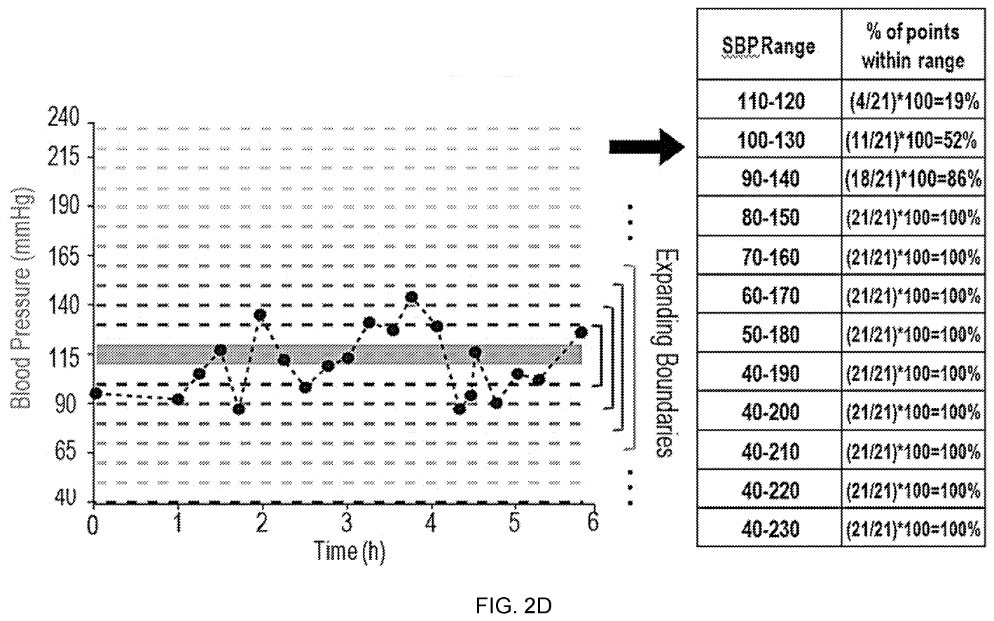

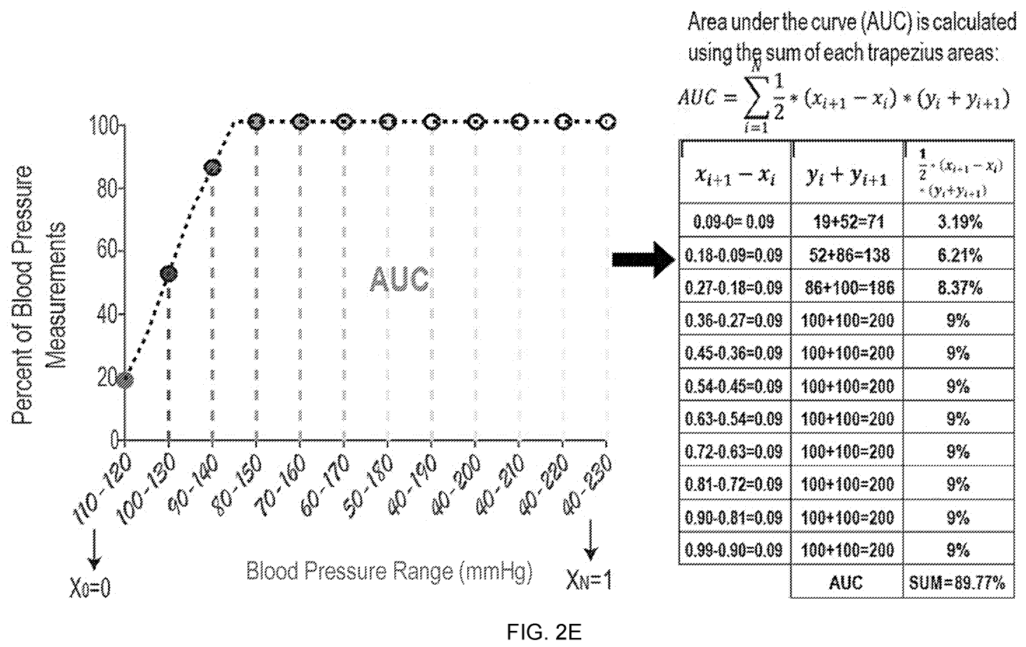

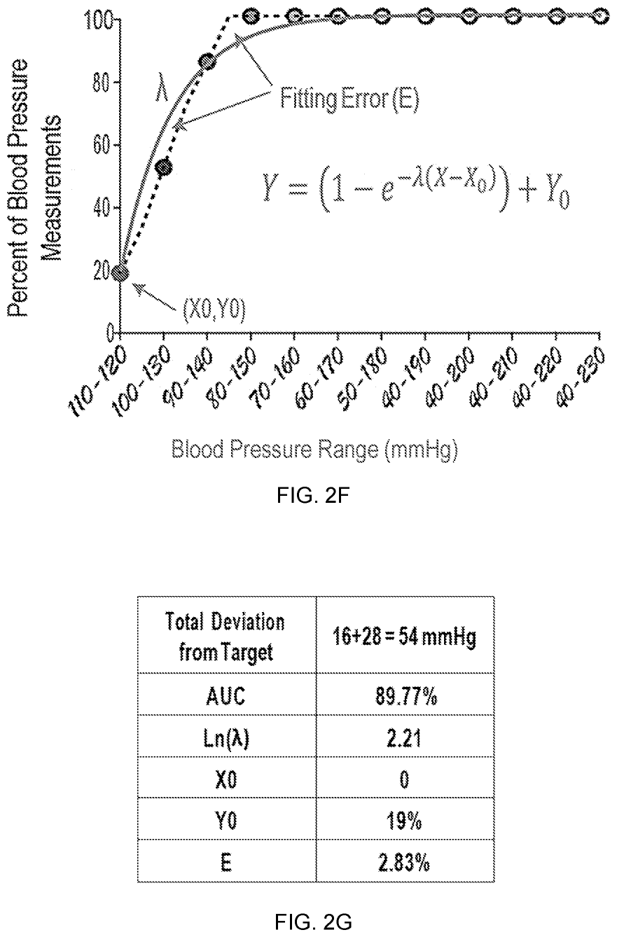

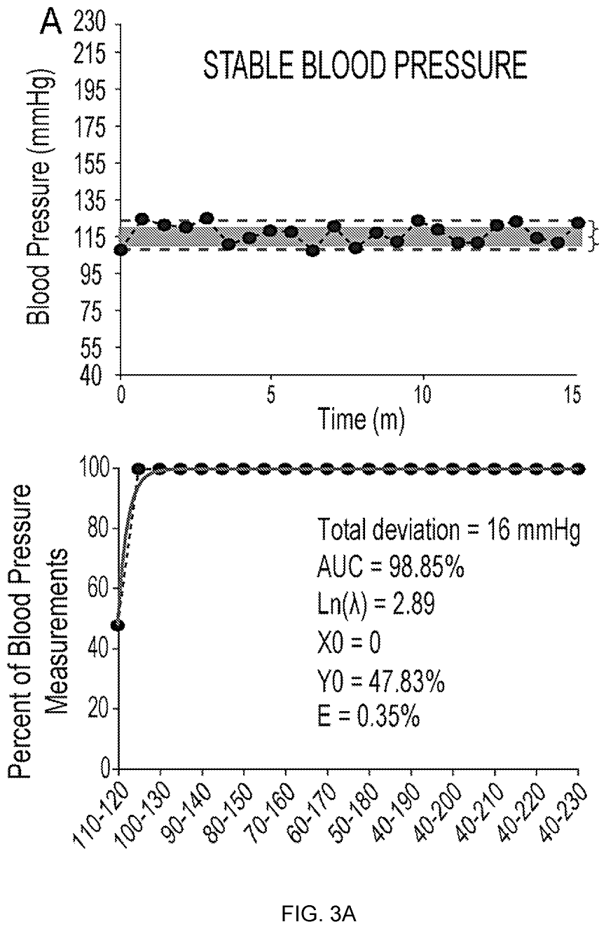

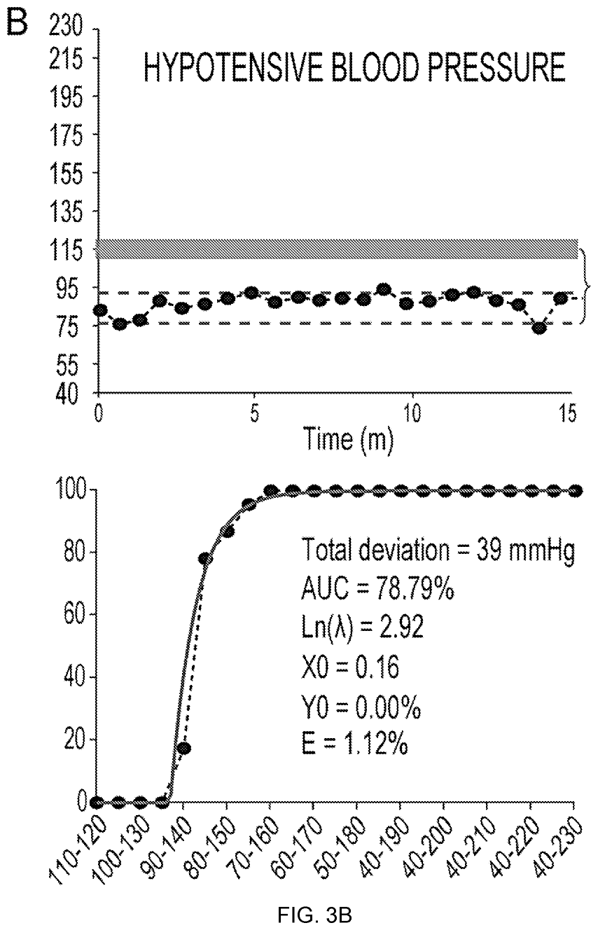

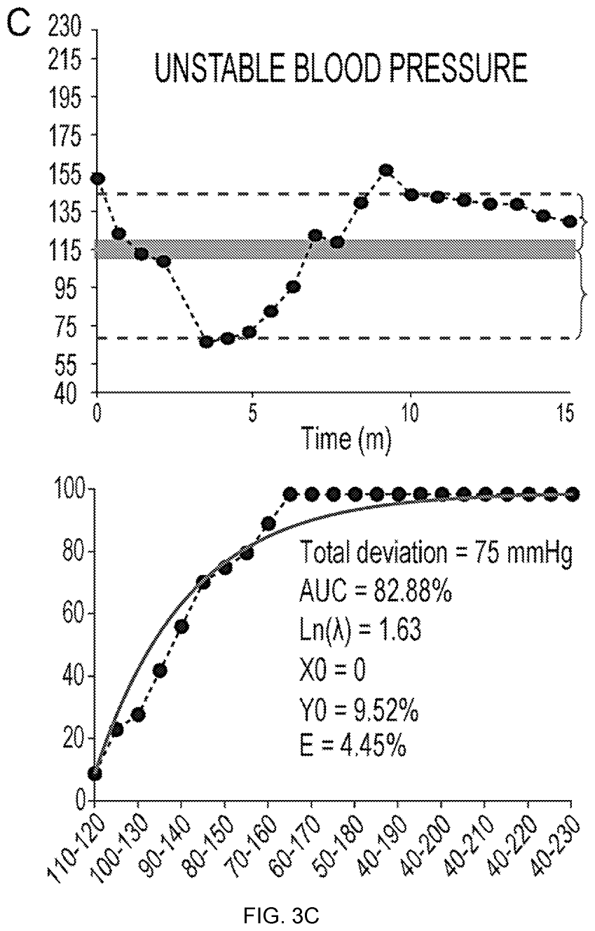

The first outcome measure, referred to as “total deviation from target,” is defined as the overall amount of deviation of the range 90% of the systolic blood pressure data points from the center of the target range (i.e., the target value, 115 mmHg). Data within the 5th (lower limit) and 95th (upper limit) percentiles was omitted to remove the effect of outliers, leaving 90% of all data points for analysis. Total deviation from target is the sum of the deviation of measurements above 115 mmHg and deviation of data points below 115 mmHg (see FIGS. 2A-2C and FIGS. 3A-3C). If all the systolic blood pressure data points only fall on one side of the 115 mmHg line, the total deviation from target is calculated as the distance between 115 mmHg and the lower limit, if the data points fall below 115 mmHg after removing outliers (see FIG. 3B), or the distance between 115 mmHg and the upper limit, if the data points fall above 115 mmHg only. While this measure quantifies the distance of the farthest, non-outlier, data points from the center of the target range, it does not describe the distribution pattern of all the data points with respect to the target range.

### Cumulative Distribution Curve

In order to quantify the distribution pattern of SBP data points with respect to the target range, a new methodology was developed based on the theory of cumulative distribution function. In this method, a cumulative distribution curve is built based on the percentage of SBP measurements within a given range, beginning with the target range (110-120 mmHg), and then by expanding the upper and lower boundaries of this range by a given expansion rate, as described below. With each expansion, the percentage of SBP measurements within each range is calculated until the final range includes all SBP values which are reasonably physiologically possible, e.g., 40-230 mmHg (FIGS. 2D and 2E). It should be noted that there is a floor-effect with this analysis: the lower range ends at 40 mmHg while the upper range increases to 230 mmHg. While the range of 40-230 mmHg is used in the referenced embodiment as the largest range of reasonably physiologically possible SBP values, in other embodiments other ranges may be used to reflect to physiological limits of SBP in live humans, or other ranges may be used with non-human subjects (e.g., dogs, cats, horses, cows, sheep, goats, chickens, etc.). The expansion rate is a parameter that defines the spatial resolution of the proposed method. In some embodiments, the expansion rate is 10 mmHg. In embodiments with higher resolution, the expansion rate may be lower value, such as, for example, 5 mmHg, 2 mmHg, or 1 mmHg. In other embodiments, higher expansion rates may be used, such as, for example, 15 mmHg or 20 mmHg. For beat-to-beat blood pressure recordings where there are many data points, it may be preferable to use an expansion rate of 1 mmHg to capture additional details about the distribution patterns of data points. For brachial blood pressure recordings (see FIGS. 2A and 2B) where fewer data points are collected, and especially when data points can be distributed widely throughout the range, the expansion rate can be set at 5 mmHg or 10 mmHg to achieve a smoother cumulative distribution curve.

### Area Under the Curve

The area under the curve (AUC) is calculated using the cumulative distribution curve. The AUC is a percentage calculated as the sum of all trapezoidal areas contained between each pair of consecutive points on the curve and the x-axis (see FIG. 2E), To calculate the AUC, the x-axis is mapped linearly and equidistant between 0 and 1 based on the selected expansion rate. The calculation formula for AUC is as following:

$$AUC = {\sum }_{i = 1}^{N} \frac{1}{2} * ( {x}_{i + 1} - {x}_{i} ) * ( {y}_{i} + {y}_{i + 1} + 1 ) \qquad Eq . 1$$

where N is the number of expansions; xᵢ and xᵢ₊₁ represent values between 0 and 1 for consecutive expanded ranges and yᵢ and yᵢ₊₁ represent consecutive percentages of the measurements within ranges xᵢ and xᵢ₊₁. An AUC of 100% indicates all observed SBP measurements are within the target range. Values less than 100% but higher than 95% indicate that the measurements are distributed very close to the target range. Lower AUC values indicate that the blood pressure data points are farther away from the target range.

### Exponential Cumulative Distribution Function

In order to quantitatively describe the shape of the cumulative distribution curve, as shown in FIG. 2F, an exponential cumulative distribution function is fit to this curve with the following equation:

$$Y = \{ \begin{matrix}( 1 - {e}^{- λ ( X - {X}_{0} )} ) + {Y}_{0} & X \geq 0 \\ 0 & X < 0\end{matrix} \qquad Eq . 2$$

and from this function five outcome measures are calculated as described below.

### Natural Logarithm of the Rate Parameter

The rate parameter (λ) in the cumulative exponential function describes the slope of the curve, with higher λ values representing steeper slope. Since the relationship between λ and the slope of the exponential curve is non-linear (i.e. small changes in λ at lower values affect the slope more than greater changes in λ at higher values) the natural logarithm of λ is used as an outcome measure to remove this nonlinearity. The natural logarithm (In) of λ can be any value between zero, indicating the shallowest slope, and 4, indicating the steepest slope. Larger values of In(λ) indicate the blood pressure data points have formed clustered distribution (either close to the target range or far away), whereas smaller values of In(λ) indicate data points have more scattered distribution either on one side (hypertension or hypotension) or both sides (unstable, having episodes of both hypertension and hypotension) of the target range.

### x- and y-axis Intercepts

The intercepts of the fitted exponential function on the x- and y-axis, (X₀, Y₀), are included as outcome measures (FIG. 2F). X₀ and Y₀ are non-negative values that can both be 0, but both values cannot be greater than 0 at the same time. If Y₀=0 and X₀>0, none of the blood pressure measurements fall within the target range of 110-120 mmHg and the value of Xo indicates the penultimate range without any blood pressure data points. If Y₀>0 and X₀=0, a percentage of data points (Y₀) fall within the target range; when Y₀=100%, all recorded data points are within the target range. If Y₀=0 and X₀=0, it means that there are no data points within the target range, but the adjacent range includes a percentage of the measurements, indicating blood pressure observations are distributed close to the target range.

It should be noted that although the rate parameter and X₀ and Y₀ are individually relevant to assessing blood pressure, together, these three outcome measures quantify the shape of the cumulative distribution curve and therefore for the statistical comparison, the rate parameter, X₀, Y₀ are combined as

$$\frac{ln ( λ )}{4} + \frac{{Y}_{0}}{100} - {X}_{0} .$$

The maximum value of In(λ) is 4 and Y₀ is a percentage and therefore by dividing each by their maximum, each parameter is normalized to a value between 0 and 1. X₀ is already between 0 and 1 (0 being the target SBP range and 1 being the broadest SBP range, as shown in FIG. 2F). Because of its inverse effect on blood pressure stability, Xo is subtracted from the sum of normalized In(λ) and Y₀. X₀ and Y₀ cannot both have positive values and therefore they either enhance (Y₀>0 and X₀=0) or reduce (Y₀=0 and X₀>0) the effect of normalized In(λ) in the combined formula.

### Fitting Error

The fitting error (E) is calculated as the mean value of absolute differences between the data points on the fitted exponential function and the cumulative distribution curve (FIG. 2F). The smallest possible E value is zero, meaning that the exponential function was a perfect fit to the cumulative distribution curve. Relatively small non-zero E values indicate that the blood pressure measurements substantially follow an exponential distribution pattern which means that data points are clustered very closely to each other (FIGS. 3A and 3B, bottom panels). Larger E values mean that the blood pressure measurements do not follow an exponential distribution pattern, i.e. data points are widely distributed on one or both sides of the target range with partial clusters (FIG. 3C, bottom panel).

The combination of these six outcome measures (total deviation, AUC, In(λ), X₀, Y₀, and E), or in some embodiments, a subset thereof, can accurately describe various distribution patterns of systolic blood pressure recordings to quantify the differences between systolic blood pressure that is stable and within normative range as shown in FIG. 3A, stable but hypotensive SBP as shown in FIG. 3B (or stable but hypertensive), and unstable SBP with episodes of hypotension and hypertension as shown in FIG. 3C.

### Validation of the Stability Measure

The six disclosed measures are validated based on the effect size (ES) to analyze discrimination (SCI vs NI) and responsiveness (supine vs sit/tilt for SCI), and based on the intra-class correlation coefficient (ICC) to evaluate the test-retest reliability (multiple assessments during screening in supine/awake for SCI).

Evaluating discrimination: The discrimination refers to the ability of the measure to distinguish groups of individuals known to be different (ability to detect inter-group differences). In this case, this measure distinguishes between individuals who have cardiovascular dysfunction and those who do not. Discrimination is evaluated with effect size. The effect size is the standardized mean difference between the two groups. It quantifies the observed difference in terms of the pooled standard deviation. The effect size is classified as tiny (<0.01), very small (0.01-0.2), small (0.2-0.5), medium (0.5-0.8), large (0.8-1.2), very large (1.2-2.0) and huge (>2.0). An effect size of 0.5 or higher is considered relevant for the purpose of discriminating between individuals with and without cardiac dysfunction.

Evaluating responsiveness: A measure is responsive if it can detect a change within individuals. During the sit-up and tilt maneuver, the measures are evaluated to determine if they can detect changes in blood pressure when moving from one position (supine) to another (sit or tilt) or from awake to sleep during 24-hour blood pressure monitoring. Responsiveness was also measured with the effect size of paired differences.

Evaluating reliability: The consistency of the outcome measures over time, i.e. the test-retest reliability, were measured as well. The test-retest reliability refers to the property of a measure to be statistically stable across time when the individuals do not experience any change. Reliability, also referred to as consistency, was evaluated using intra-class correlation coefficient (ICC) and standard error of measurement (SEM). The ICC is calculated using mixed models. Let σᵣ² be the variance of the random effect and σₑ² be the variance of the model error term. The ICC is calculated by the formula:

$$ICC = \frac{{σ}_{r}^{2}}{{σ}_{r}^{2} + {σ}_{e}^{2}} .$$

Reliability has been classified as poor (0-0.25), fair (0.25-0.5), moderate (0.5-0.75), good (0.75-0.90) and excellent (>0.90).

The SEM is also a clinically useful metric which allows practitioners to make inferences about individual changes in a test. SEM values are in the same units as the units of the variable being analyzed.

FIGS. 3A-3C show three examples of SBP recordings with stable and two different unstable patterns and the corresponding outcome measures for each example.

### Sensitivity Analysis: Effects of Expansion Rate, Averaging and Downsampling on Proposed Outcome Measures

In analysis of continuous blood pressure recordings, particularly those obtained from the finger, it is routine to average the data points over a short period of time to remove clinically insignificant variabilities in the data that occur due to the acquisition methodology. Downsampling the data points is also sometimes used to remove excessive number of data points. The definition of the stability measures also involves the choice of the expansion rate parameter from the target range 110-120 mmHg. A sensitivity analysis was performed to evaluate whether the results obtained would change based on a different choice of these parameters.

Data averaging: Analyzing systolic blood pressure data may use beat-to-beat data or averaging over a selected time interval. To evaluate any effect on the results found, multiple averaging schemas were performed (5, 10, 15, . . . , 60 seconds). Stability measures were calculated for each averaging and were compared to those obtained with no averaging (beat-to-beat data).

Downsampling: Downsampling refers to a systematic choice of fewer points from the sample. The effect of picking every nth systolic blood pressure value on the obtained stability measure was evaluated by varying n from 2 (every other data point) to 20 (every 20^{th} data point) and calculating the corresponding stability measures and comparing them to what is obtained when every measurement it considered. FIGS. 3A-3C depict data downsampled by 50 points for visualization purposes, but downsampling by greater than 20 points is not recommended for analysis of blood pressure data.

Expansion rate: To calculate the area under the curve, boundaries are expanded symmetrically from the target range 110-120 mmHg with equal jumps (at least, until the range expands to 40-190 mmHg; afterwards only the top end of the range increases) and the percentage of values falling in that range are calculated. The effects that the choice of R would have on the calculated outcome measures were evaluated by comparing the values found when R=2, 3, 4, . . . 10 mmHg and comparing to R=1 mmHg for beat-to-beat blood pressure recordings.

### Statistical Analysis

Participants characteristics (demographics and injury details) were summarized using mean and standard deviation (SD) for continuous descriptors, and frequency count and percentage for categorical descriptors.

Discrimination was evaluated between groups known to have physiologically different responses, i.e., comparing non-injured vs individuals with SCI in the Sit Up Test, 70° tilt test, and 24 Hour BP Monitoring assessments. The measure used is effect size using Cohen's d (for similar sample sizes) or Hedges' g (for different sample sizes) formulas, calculated as the standardized difference between the mean values of the non-injured and SCI groups in sitting and tilt positions and during awake time from 24-hour recordings. The systolic blood pressure data from Sit-up test and 70° Tilt test was used to evaluate responsiveness given the known changes that occur in individuals with SCI from supine to sitting or from supine to tilt. Paired changes, supine to sitting or tilt, were calculated for each individual and used to calculate the effect size. The ICC value used to evaluate reliability was obtained from mixed models using data recorded in supine from SCI participants who have had two measurements during the screening phase without any changes to their day-to-day life and confirmed no change in their cardiovascular function in between. These models included a random intercept for each participant. The variance of the residuals σᵣ² and the variance of the random intercepts σₑ² were obtained and used in the formula of the ICC as the estimates for the variance of the error term and random effect, respectively.

The evaluation of the effects of averaging, downsampling and Expansion Rate were performed using paired t-test of the stability measure values resulting from different scenarios described in the earlier “Effects of expansion rate, averaging and downsampling on measures' outcomes” section. All tests were 2-sided with a significance level of 0.05.

### RESULTS

### Study Participants

Sit up test: The non-injured group of 48 individuals were 40±13 years old and 67% were males. The combined SCI group (n=45) was composed of 37±11 years old individuals, 27±12 years after injury, 84% males with 78% cervical injuries distributed across the American Spinal Injury Association Impairment Scale (AIS) grades A-D.

70° tilt test: The 9 non-injured participants were 31±11 years old at the time of experiments and 67% were males. The 24 SCI participants were 39±11 years old, 75% males, 71% cervical injuries, 50% AIS A, 38% AIS B and 13% AIS C, and 11±8 years after injury. A subset of SCI (n=10, all cervical injuries) was used for test-retest reliability (80% male, 33±13 years old, 7±4 years post injury).

24-hour blood pressure monitoring: Of the twelve non-injured individuals included in the 24-hour blood pressure monitoring, 58% males and 27±5 years old at the time of assessment. The individuals with SCI were divided into two groups: the screening group (SCI-G1: n=13) and the scES implanted group (SCI-G2: n=9); during the 24-hour blood pressure monitoring assessment, stimulation remained OFF throughout the recording. The SCI-G1 were 54% males and 37±14 years old at the time of assessment and 9±6 years post injury. The SCI-G2 were 89% males and 31±9 years old at the time of screening and 7±4 years post injury. Only SCI-G1 data was used for discrimination and responsiveness analysis to avoid the interference of possible effects of scES on daily blood pressure in SCI-G2. A subgroup of SCI-G1 and all participants data in SCI-G2 were used for test-retest analysis (n=15). The test-retest blood pressure recordings for SCI-G2 were performed with scES off and prior to scES-cardiovascular training, therefore no effects from the stimulation were expected.

6-hour blood pressure monitoring for scES effects: Individuals with SCI without intervention for cardiovascular stability (n=15) were 60% male, 36±15 years old, 10±9 years post injury; individuals with the scES implant targeting cardiovascular function (n=9) were 78% male, 31±6 years old, 7±3 years post injury.

### Validation of Blood Pressure Stability Outcome Measures

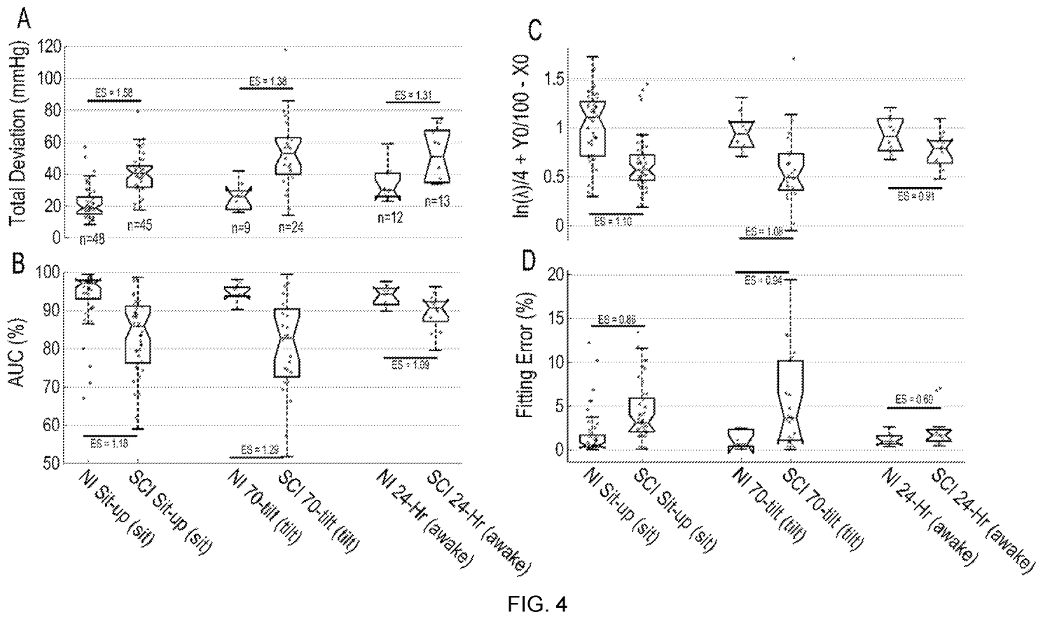

Results of the validation measures for the proposed blood pressure stability toolset are presented in FIGS. 4-6 and Table 1. The validation analysis demonstrates all proposed outcome measures for blood pressure stability are discriminatory, responsive, and reliable based on data recorded during sit-up test, 70° tilt test, and 24-hour blood pressure monitoring.

Table 1: Validation characteristics of the proposed blood pressure stability outcome measures. Reliability, discrimination and responsiveness were evaluated. Responsiveness and discrimination were evaluated using effect size (ES). ES is classified as tiny (<0.01), very small (0.01-0.2), small (0.2-0.5), medium (0.5-0.8), large (0.8-1.2), very large (1.2-2.0) and huge (>2.0). An ES of 0.5 or larger has been classified as relevant. Reliability was measure with the intraclass correlation coefficient (ICC) and standard error of measurement (SEM). ICC is classified as poor (0-0.25), fair (0.25-0.5), moderate (0.5-0.75), good (0.75-0.90) and excellent (>0.90). A measure with ICC of 0.5 is considered to be reliable. Estimate values above this 0.5 represent a discriminatory, responsive or reliable measure. SE: Standard Error.

| | | | | | | Evaluation | | |
|---|---|---|---|---|---|---|---|---|
| | | | | n | Mean ± SE | measure | Classification | Comment |
| Discrimination | Total | Sit Up | NI sitting | 48 | 22 ± 1.50 | ES: 1.58 | Very large | Discriminatory |
| | deviation | Test | position | | | | effect | |
| | from 115 | | SCI sitting | 45 | 40 ± 1.88 | | | |
| | mmHg | | position | | | | | |
| | | 70-degree | NI tilt | 9 | 25 ± 2.84 | ES: 1.38 | Very large | Discriminatory |
| | | Tilt | position | | | | effect | |
| | | Maneuver | SCI tilt | 24 | 53 ± 4.70 | | | |
| | | | position | | | | | |
| | | 24 Hr BP | NI awake | 12 | 33 ± 3.02 | ES: 1.31 | Very large | Discriminatory |
| | | Monitoring | SCI awake | 13 | 52 ± 4.69 | | effect | |
| | Area | Sit Up | NI sitting | 48 | 94 ± 1.04 | ES: 1.18 | Large effect | Discriminatory |
| | Under | Test | position | | | | | |
| | the curve | | SCI sitting | 45 | 83 ± 1.53 | | | |
| | | | position | | | | | |
| | | 70-degree | NI tilt | 9 | 95 ± 0.77 | ES: 1.29 | Very large | Discriminatory |
| | | Tilt | position | | | | effect | |
| | | Maneuver | SCI tilt | 24 | 81 ± 2.55 | | | |
| | | | position | | | | | |
| | | 24 Hr BP | NI awake | 12 | 94 ± 0.72 | ES: 1.09 | Large effect | Discriminatory |
| | | Monitoring | SCI awake | 13 | 90 ± 1.30 | | | |
| | $\frac{ln ( λ )}{4} + \frac{Y 0}{100}$ | Sit Up Test | NI sitting position SCI sitting position | 48 45 | 1.03 ± 0.05 0.68 ± 0.04 | ES: 1.10 | Large effect | Discriminatory |
| | | 70-degree | NI tilt | 9 | 0.95 ± 0.07 | ES: 1.08 | Large effect | Discriminatory |
| | | Tilt | position | | | | | |
| | | Maneuver | SCI tilt | 24 | 0.58 ± 0.08 | | | |
| | | | position | | | | | |
| | | 24 Hr BP | NI awake | 12 | 0.93 ± 0.05 | ES: 0.91 | Large effect | Discriminatory |
| | | Monitoring | SCI awake | 13 | 0.77 ± 0.05 | | | |
| | Exponential | Sit Up | NI sitting | 48 | 1.68 ± 0.37 | ES: 0.86 | Large effect | Discriminatory |
| | Fitting | Test | position | | | | | |
| | Error | | SCI sitting | 45 | 4.31 ± 0.51 | | | |
| | | | position | | | | | |
| | | 70-degree | NI tilt | 9 | 1.17 ± 0.33 | ES: 0.94 | Large effect | Discriminatory |
| | | Tilt | position | | | | | |
| | | Maneuver | SCI tilt | 24 | 5.45 ± 1.07 | | | |
| | | | position | | | | | |
| | | 24 Hr BP | NI awake | 12 | 1.21 ± 0.21 | ES: 0.69 | Medium effect | Discriminatory |
| | | Monitoring | SCI awake | 13 | 2.33 ± 0.59 | | | |
| Responsiveness | Total | Sit Up | SCI supine | 45 | 23 ± 1.79 | ES: 1.40 | Very large | Responsive |
| | deviation | Test | position | | | | effect | |
| | from 115 | | SCI sitting | 45 | 40 ± 1.88 | | | |
| | mmHg | | position | | | | | |
| | | 70-degree | SCI supine | 24 | 21 ± 2.03 | ES: 1.82 | Very large | Responsive |
| | | Tilt | position | | | | effect | |
| | | Maneuver | SCI tilt | 24 | 53 ± 4.7 | | | |
| | | | position | | | | | |
| | Area | Sit Up | SCI supine | 45 | 93 ± 1.12 | ES: 1.08 | Large effect | Responsive |
| | Under | Test | position | | | | | |
| | the curve | | SCI sitting | 45 | 83 ± 1.53 | | | |
| | | | position | | | | | |
| | | 70-degree | SCI supine | 24 | 92 ± 1.52 | ES: 1.07 | Large effect | Responsive |
| | | Tilt | position | | | | | |
| | | Maneuver | SCI tilt | 24 | 81 ± 2.55 | | | |
| | | | position | | | | | |
| | $\frac{ln ( λ )}{4} + \frac{Y 0}{100}$ | Sit Up Test | SCI supine position SCI sitting position | 45 45 | 1.02 ± 0.06 0.64 ± 0.04 | ES: 1.05 | Large effect | Responsive |
| | | 70-degree | SCI supine | 24 | 0.91 ± 0.09 | ES: 0.81 | Large effect | Responsive |
| | | Tilt | position | | | | | |
| | | Maneuver | SCI tilt | 24 | 0.58 ± 0.08 | | | |
| | | | position | | | | | |
| | Exponential | Sit Up | SCI supine | 45 | 1.78 ± 0.33 | ES: 0.88 | Large effect | Responsive |
| | Fitting | Test | position | | | | | |
| | Error | | SCI sitting | 45 | 4.31 ± 0.51 | | | |
| | | | position | | | | | |
| | | 70-degree | SCI supine | 24 | 2.30 ± 0.33 | ES: 0.81 | Large effect | Responsive |
| | | Tilt | position | | | | | |
| | | Maneuver | SCI tilt | 24 | 5.45 ± 1.07 | | | |
| | | | position | | | | | |
| Test-retest | Total | Sit Up | SCI Supine | 8 | 18 ± 2.70 | ICC: 0.96 | Excellent | Reliable |
| reliability | deviation | Test | Day 1 | | | | Reliability | |
| | from 115 | | SCI Supine | 8 | 18 ± 2.43 | SEM: 1.44 | | |
| | mmHg | | Day 2 | | | | | |
| | | 70-degree | SCI Supine | 10 | 15 ± 1.32 | ICC: 0.81 | Good | Reliable |
| | | Tilt | Day 1 | | | | Reliability | |
| | | Maneuver | SCI Supine | 10 | 16 ± 1.75 | SEM: 2.1 | | |
| | | | Day 2 | | | | | |
| | | 24 Hr BP | SCI awake | 15 | 52 ± 3.37 | ICC: 0.64 | Moderate | Reliable |
| | | Monitoring | Day 1 | | | | Reliability | |
| | | | SCI awake | 15 | 53 ± 3.30 | SEM: 7.99 | | |
| | | | Day 2 | | | | | |
| | Area | Sit Up | SCI Supine | 8 | 94 ± 2.12 | ICC: 0.97 | Excellent | Reliable |
| | Under | Test | Day 1 | | | | Reliability | |
| | the curve | | SCI Supine | 8 | 94 ± 2.26 | SEM: 1.05 | | |
| | | | Day 2 | | | | | |
| | | 70-degree | SCI Supine | 10 | 95 ± 1.37 | ICC: 0.91 | Excellent | Reliable |
| | | Tilt | Day 1 | | | | Reliability | |
| | | Maneuver | SCI Supine | 10 | 95 ± 1.21 | SEM: 1.30 | | |
| | | | Day 2 | | | | | |
| | | 24 Hr BP | SCI awake | 15 | 88 ± 1.98 | ICC: 0.59 | Moderate | Reliable |
| | | Monitoring | Day 1 | | | | Reliability | |
| | | | SCI awake | 15 | 88 ± 1.24 | SEM: 4.18 | | |
| | | | Day 2 | | | | | |
| | $\frac{ln ( λ )}{4} + \frac{Y 0}{100}$ | Sit Up Test | SCI Supine Day 1 SCI Supine Day 2 | 8 8 | 1.06 ± 0.17 1.08 ± 0.18 | ICC: 0.97 SEM: 0.09 | Excellent Reliability | Reliable |
| | | 70-degree | SCI Supine | 10 | 1.00 ± 0.11 | ICC: 0.87 | Good | Reliable |
| | | Tilt | Day 1 | | | | Reliability | |
| | | Maneuver | SCI Supine | 10 | 0.99 ± 0.11 | SEM: 0.13 | | |
| | | | Day 2 | | | | | |
| | | 24 Hr BP | SCI awake | 15 | 0.72 ± 0.05 | ICC: 0.54 | Moderate | Reliable |
| | | Monitoring | Day 1 | | | | Reliability | |
| | | | SCI awake | 15 | 0.70 ± 0.04 | SEM: 0.12 | | |
| | | | Day 2 | | | | | |
| | Exponential | Sit Up | SCI Supine | 8 | 1.69 ± 0.53 | ICC: 0.97 | Excellent | Reliable |
| | Fitting | Test | Day 1 | | | | Reliability | |
| | Error | | SCI Supine | 8 | 1.54 ± 0.52 | SEM: 0.27 | | |
| | | | Day 2 | | | | | |
| | | 70-degree | SCI Supine | 10 | 1.44 ± 0.28 | ICC: 0.65 | Moderate | Reliable |
| | | Tilt | Day 1 | | | | Reliability | |
| | | Maneuver | SCI Supine | 10 | 1.80 ± 0.38 | SEM: 0.61 | | |
| | | | Day 2 | | | | | |
| | | 24 Hr BP | SCI awake | 15 | 2.21 ± 0.45 | ICC: 0.73 | Moderate | Reliable |
| | | Monitoring | Day 1 | | | | Reliability | |
| | | | SCI awake | 15 | 3.02 ± 0.58 | SEM: 0.94 | | |
| | | | Day 2 | | | | | |

Details of discrimination analysis are depicted in panels A-D of FIG. 4. While all four outcome measures depicted in this figure demonstrate the difference between non-injured and SCI, total deviation from the 115 mmHg target value has the greatest Effect Size (ES) values; this is expected, since this measure depends on the most (non-outlier) extreme blood pressure values that individuals experience during the perturbation tests and daily variability of their blood pressure. The AUC, the integrated value for In(λ), Y₀, and X₀ demonstrate the difference in variability of systolic blood pressure with respect to the target range between individuals with SCI and non-injured individuals. The fitting error demonstrates how closely the distribution of systolic blood pressure data points follow an exponential cumulative curve.

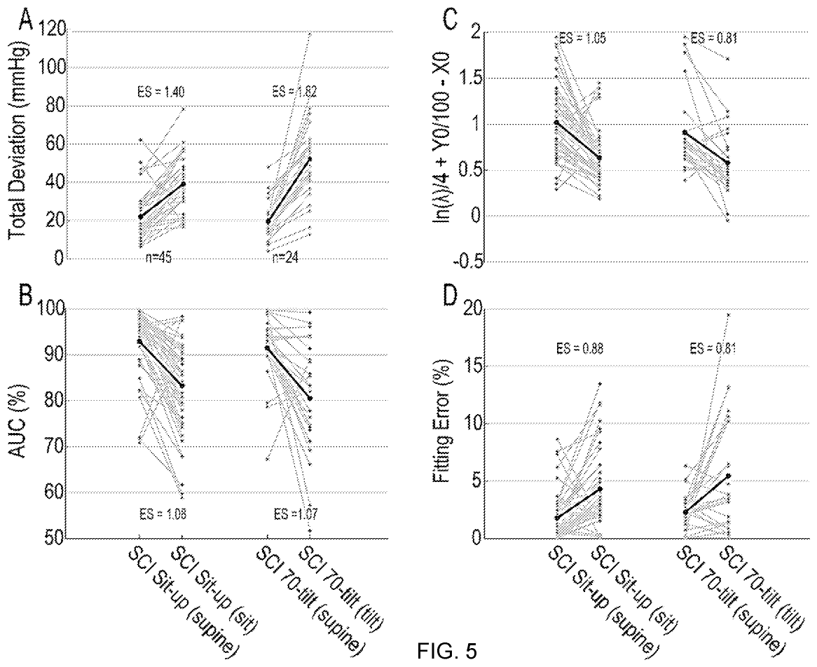

The responsiveness analysis depicted in panels A-D of FIG. 5 details the differences within individuals with SCI in two different positions (supine versus sit and supine versus 70° tilt). Similar to the discrimination analysis, total deviation from 115 mmHg has greatest ES values compared with other outcome measures, but all outcome measures demonstrate differences in systolic blood pressure distribution within and around the target range between different positions.

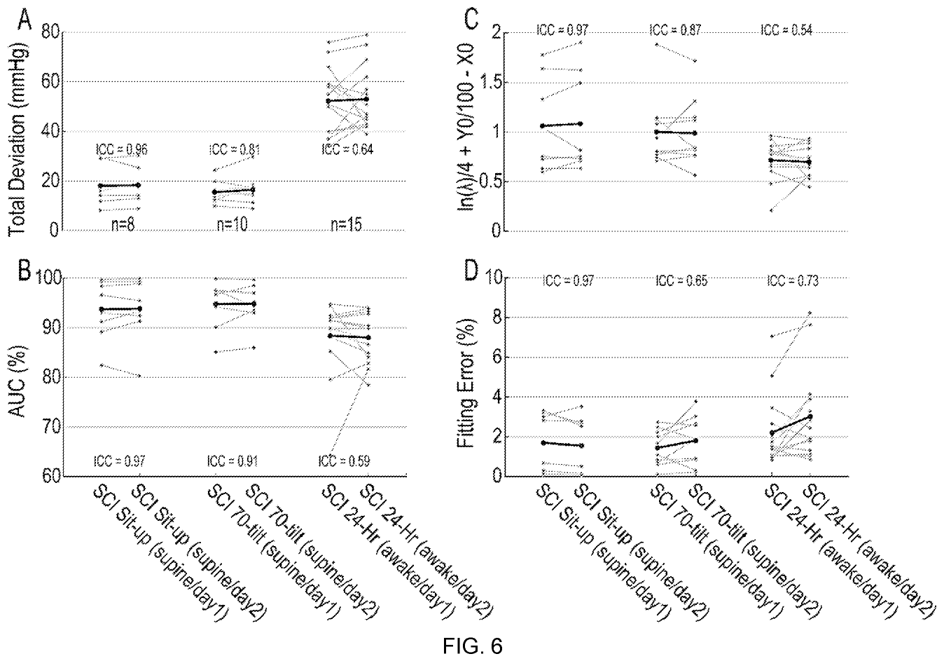

In test-retest analysis performed on repeated blood pressure recordings depicted in panels A-D of FIG. 6, all outcome measures demonstrate no significant difference between the two perturbation tests or 24-hour tests across individuals with chronic SCI.

Table 1 presents the details of discrimination, responsiveness and test-retest analysis with the classification of the evaluation measures.

### Effects of scES Intervention on Cardiovascular Function

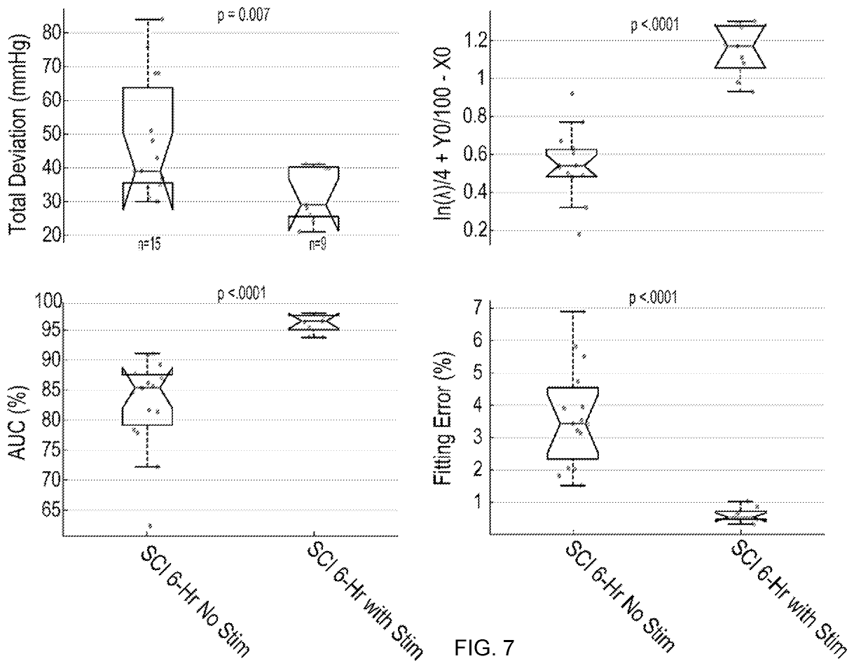

The results of the proposed stability outcome measures were compared between two groups of individuals with chronic SCI, one group with and the other group without a scES intervention that targets cardiovascular regulation. The results are depicted in FIG. 7, illustrating all outcome measures disclosed herein can demonstrate the improvement in the blood pressure stability in the presence of scES and the differences between the two groups are statistically significant.

### Sensitivity Analysis on the Choice of Averaging, Downsampling and Expansion Rate

As indicated in Table 2, averaging data was found to have a significant effect on the resulting Stability Measure outcomes, as indicated by p values of <0.05. As indicated in Table 3, downsampling did not have a significant effect on the AUC, total deviation from 115 mmHg, and SBP range containing 90% of the data for most choices, but higher levels of downsampling significantly altered the values obtained for the curve corresponding exponential function filling error. As indicated in Table 4, the expansion rate had a significant effect on the AUC, and the fitting error but minor effect on the curve corresponding to exponential function parameter

$$\frac{ln ( λ )}{4} + \frac{{Y}_{0}}{100} - {X}_{0} .$$

Accordingly, when analyzing data, particular caution should be used when averaging data or using expansion rates greater than 1 mmHg.

TABLE 2
Effect of averaging on the stability measure outcomes.

| | AUC | | Total deviation from 115 | | $\frac{ln ( λ )}{4} + \frac{Y 0}{100} - X 0$ | | Exp Fitting error | |
|---|---|---|---|---|---|---|---|---|
| | Mean | | Mean | | Mean | | Mean | |
| | (95% Cl) | p* | (95% Cl) | p* | (95% Cl) | p* | (95% Cl) | p* |
| No | 80.94 | | | | 1.26 (1.14, | | | |
| averaging | (75.86, | | 52.5 (43.02, | | 1.39) | | 5.43 (3.31, | |
| | 86.01) | | 61.99) | | | | 7.56) | |
| | 81.17 | | 50.18 | | 1.29 (1.16, | 0.0033 | | |
| Every 5 s | (76.08, | 0.1382 | (40.43, | | 1.41) | | 4.71 (3.04, | 0.0391 |
| | 86.26) | | 59.93) | 0.0114 | | | 6.39) | |
| | 81.28 | | 49.29 | | 1.29 (1.17, | 0.0006 | | |
| Every 10 | (76.17, | 0.0322 | (39.77, | | 1.42) | | 4.49 (2.95, | |
| s | 86.39) | | 58.82) | 0.0005 | | | 6.03) | 0.007 |
| | 81.34 | | | | 1.3 (1.18, | <.0001 | | |
| Every 15 | (76.2, | 0.0123 | 47.7 (38.35, | | 1.43) | | 4.27 (2.82, | 0.0009 |
| s | 86.48) | | 57.04) | <.0001 | | | 5.71) | |
| | 81.66 | | 46.29 | | 1.32 (1.2, | <.0001 | | |
| Every 20 | (76.59, | <.0001 | (37.35, | | 1.45) | | 4.21 (2.88, | 0.0005 |
| s | 86.73) | | 55.23) | <.0001 | | | 5.54) | |
| | 81.42 | | 46.15 | | 1.31 (1.19, | <.0001 | | |
| Every 25 | (76.22, | 0.0027 | (37.16, | | 1.44) | | 3.93 (2.68, | <.0001 |
| s | 86.62) | | 55.15) | <.0001 | | | 5.18) | |
| | 81.61 | | | | 1.33 (1.2, | <.0001 | | |
| Every 30 | (76.48, | <.0001 | 45.69 | | 1.45) | | 3.82 (2.61, | <.0001 |
| s | 86.75) | | (36.68, 54.7) | <.0001 | | | 5.03) | |
| | 81.98 | | 45.21 | | 1.33 (1.21, | <.0001 | | |
| Every 35 | (76.67, | <.0001 | (35.55, | | 1.46) | | 3.8 (2.64, | <.0001 |
| s | 87.29) | | 54.86) | <.0001 | | | 4.96) | |
| | 82.05 | | | | 1.33 (1.2, | <.0001 | | |
| Every 40 | (76.82, | <.0001 | 43.97 | | 1.45) | | 3.75 (2.64, | <.0001 |
| s | 87.28) | | (34.73, 53.2) | <.0001 | | | 4.86) | |
| | 81.97 | | 44.43 | | 1.34 (1.21, | <.0001 | | |
| Every 45 | (76.61, | <.0001 | (35.17, | | 1.46) | | 3.57 (2.54, | <.0001 |
| s | 87.33) | | 53.69) | <.0001 | | | 4.6) | |
| | 82.01 | | 43.51 | | 1.34 (1.22, | <.0001 | | |
| Every 50 | (76.64, | <.0001 | (34.74, | | 1.46) | | 3.56 (2.53, | <.0001 |
| s | 87.39) | | 52.29) | <.0001 | | | 4.58) | |
| | 82.05 | | 42.67 | | 1.34 (1.22, | <.0001 | | |
| Every 55 | (76.76, | <.0001 | (34.29, | | 1.47) | | 3.57 (2.56, | <.0001 |
| s | 87.35) | | 51.06) | <.0001 | | | 4.59) | |
| | 82.16 | | | | 1.35 (1.23, | <.0001 | | |
| Every 60 | (76.89, | <.0001 | 41.5 (33.32, | | 1.47) | | 3.2 (2.31, | <.0001 |
| s | 87.43) | | 49.69) <.0001 | | | | 4.1) | |

*Comparing downsampling schemas with including every data point

TABLE 3
Effect of downsampling the proposed stability measure
outcomes

| | AUC | | Total deviation from 115 | | $\frac{ln ( λ )}{4} + \frac{Y 0}{100} - X 0$ | | Exp Fitting error | |
|---|---|---|---|---|---|---|---|---|
| | Mean | | Mean | | Mean | | Mean | |
| | (95% Cl) | p* | (95% Cl) | p* | (95% Cl) | p* | (95% Cl) | p* |
| Every | 80.94 | | 52.5 | | 1.26 (1.14, | | 5.43 | |
| point | (75.86, | | (43.02, | | 1.39) | | (3.31, | |
| | 86.01) | | 61.99) | | | | 7.56) | |
| Every | 80.96 | | 52.54 | | 1.26 (1.14, | 0.809 | 5.32 | |
| other | (75.88, | | (42.99, | | 1.39) | | (3.26, | |
| point | 86.03) | 0.882 | 62.08) 0.9584 | | | | 7.37) | 0.538 |
| Every | | | 52.34 | | 1.26 (1.13, | 0.8734 | 5.17 | |
| 3rd | 80.93 | | (42.89, | | 1.39) | | (3.15, | |
| point | (75.86, 86) | 0.9244 | 61.79) 0.782 | | | | 7.19) | 0.1555 |
| Every | 80.99 | | 52.49 | | 1.26 (1.14, | 0.6685 | 5.16 | |
| 4th | (75.93, | | (42.93, | | 1.39) | | (3.14, | |
| point | 86.06) | 0.6244 | 62.05) 0.9827 | | | | 7.19) | 0.1507 |
| Every | 80.96 | | 52.31 | | 1.27 (1.14, | 0.4076 | 5.25 | |
| 5th | (75.89, | | (42.76, | | 1.39) | | (3.23, | |
| point | 86.03) | 0.8408 | 61.86) 0.7478 | | | | 7.27) | 0.3262 |
| Every | 80.97 | | 52.24 | | 1.27 (1.14, | 0.2226 | 5.15 | |
| 6th | (75.93, | | (42.59, | | 1.4) | | (3.17, | |
| point | 86.01) | 0.7743 | 61.89) 0.6582 | | | | 7.14) | 0.1348 |
| Every | 80.93 | | | | 1.26 (1.14, | 0.9821 | 5.06 | |
| 7th | (75.89, | | 52.23 | | 1.39) | | (3.06, | |
| point | 85.96) | 0.9287 | (42.9, 61.57) | 0.6496 | | | 7.06) | 0.046 |
| Every | 80.97 | | 52.33 | | 1.26 (1.14, | 0.7568 | 5.19 | |
| 8th | (75.91, | | (42.69, | | 1.39) | | (3.14, | |
| point | 86.04) | 0.7531 | 61.98) 0.7759 | | | | 7.23) | 0.1883 |
| Every | 80.88 | | 51.93 | | 1.26 (1.14, | 0.9378 | 5.07 | |
| 9th | (75.81, | | (42.45, | | 1.39) | | (3.11, | |
| point | 85.95) | 0.6176 | 61.41) | 0.3346 | | | 7.03) | 0.0535 |
| Every | 80.9 | | 52.46 | | 1.27 (1.14, | 0.5266 | 5.24 | |
| 10th | (75.82, | | (42.81, | | 1.39) | | (3.23, | |
| point | 85.98) | 0.7311 | 62.11) 0.9437 | | | | 7.26) | 0.3092 |
| Every | 80.97 | | 51.03 | | 1.26 (1.14, | 0.7244 | 5.05 | |
| 11th | (75.86, | | (41.79, | | 1.39) | | (3.07, | |
| point | 86.09) | 0.7638 | 60.26) 0.0136 | | | | 7.03) | 0.0404 |
| Every | | | 52.25 | | 1.27 (1.14, | 0.1761 | 4.97 | |
| 12th | 80.97 | | (42.76, | | 1.4) | | (3.03, | |
| point | (75.93, 86) | 0.8112 | 61.73) | 0.668 | | | 6.92) | 0.0143 |
| Every | 80.74 | | 51.37 | | 1.26 (1.13, | 0.6943 | 5 | |
| 13th | (75.65, | | (42.51, | | 1.38) | | (3.05, | |
| point | 85.83) | 0.0843 | 60.24) 0.0589 | | | | 6.96) | 0.0219 |
| Every | | | 52.61 | | 1.27 (1.14, | 0.2909 | 4.64 | |
| 14th | 80.93 | | (43.38, | | 1.39) | | (2.84, | |
| point | (75.95, 85.9) | 0.9236 | 61.84) 0.8569 | | | | 6.44) | <. 0001 |
| Every | 80.79 | | 51.74 | | 1.27 (1.14, | 0.351 | 4.99 | |
| 15th | (75.73, | | (42.38, | | 1.39) | | (3.06, | |
| point | 85.84) | 0.2021 | 61.11) 0.2025 | | | | 6.92) | 0.0178 |
| Every | 80.83 | | 52.09 | | 1.27 (1.15, | 0.1389 | 4.9 | |
| 16th | (75.81, | | (42.18, | | 1.4) | | (3.08, | |
| point | 85.84) | 0.3363 | 62.01) | 0.4902 | | | 6.71) | 0.0043 |
| Every | 80.62 | | 52.05 | | 1.26 (1.13, | 0.6483 | 4.87 | |
| 17th | (75.46, | | (43.15, | | 1.38) | | (3.01, | |
| point | 85.79) | 0.0074 | 60.94) 0.4423 | | | | 6.72) | 0.0026 |
| Every | 80.71 | | 50.94 | | 1.27 (1.14, | 0.2958 | 5 | |
| 18th | (75.68, | | (41.93, | | 1.39) | | (3.11, | |
| point | 85.73) | 0.0497 | 59.96) | 0.0092 | | | 6.88) | 0.0203 |
| Every | 80.76 | | 51.91 | | 1.26 (1.13, | 0.883 | 5.03 | |
| 19th | (75.78, | | (43.17, | | 1.39) | | (3.14, | |
| point | 85.74) | 0.123 | 60.65) | 0.3212 | | | 6.92) | 0.0307 |
| Every | | | 51.18 | | 1.27 (1.14, | 0.2349 | 5.03 | |
| 20th | 80.87 | | (42.09, | | 1.4) | | (3.13, | |
| point | (75.8, 85.94) | 0.554 | 60.27) | 0.0269 | | | 6.92) | 0.0293 |

*Comparing downsampling schemas with including every data point

TABLE 4
Effect of expansion rate on the proposed stability measure
outcomes

| | AUC | | $\frac{ln ( λ )}{4} + \frac{Y 0}{100} - X 0$ | | Exp Fitting error | |
|---|---|---|---|---|---|---|
| | Mean (95% Cl) | p* | Mean (95% Cl) | p* | Mean (95% Cl) | p* |
| Expanding | 80.94 (75.86, | | | | | |
| 1 mmHg | 86.01) | | 1.26 (1.12, 1.4) | | 5.43 (3.31, 7.56) | |
| Expanding | | | | | | |
| 2mmHg | 80.93 (75.86, 86) | 0.9659 | 1.26 (1.12, 1.4) | 0.9039 | 5.39 (3.29, 7.49) | 0.7 |
| Expanding | 80.58 (75.42, | | | | | |
| 3mmHg | 85.74) | 0.0045 | 1.25 (1.11, 1.39) | 0.3698 | 5.31 (3.23, 7.39) | 0.3117 |
| Expanding | 80.56 (75.41, | | | | | |
| 4mmHg | 85.72) | 0.0026 | 1.25 (1.11, 1.39) | 0.5422 | 5.25 (3.18, 7.33) | 0.1338 |
| Expanding | 80.89 (75.83, | | | | | |
| 5mmHg | 85.95) | 0.7147 | 1.27 (1.13, 1.41) | 0.7722 | 5.07 (3.07, 7.08) | 0.003 |
| Expanding | | | | | | |
| 6mmHg | 79.4 (73.97, 84.83) | <.0001 | 1.21 (1.07, 1.35) | 0.0007 | 5.1 (3.12, 7.09) | 0.0065 |
| Expanding | 79.95 (74.68, | | | | | |
| 7 mmHg | 85.22) | <.0001 | 1.23 (1.09, 1.37) | 0.0643 | 4.84 (2.91, 6.76) | <.0001 |
| Expanding | 79.73 (74.42, | | | | | |
| 8mmHg | 85.04) | <.0001 | 1.22 (1.08, 1.36) | 0.0104 | 4.98 (3.01, 6.95) | 0.0002 |
| Expanding | 78.73 (73.19, | | | | | |
| 9mmHg | 84.27) | <.0001 | 1.16 (1.02, 1.3) | <.0001 | 4.93 (2.92, 6.94) | <.0001 |
| Expanding | 80.74 (75.74, | | | | | |
| 10mmHg | 85.75) | 0.1162 | 1.27 (1.13, 1.41) | 0.669 | 4.54 (2.64, 6.44) | <.0001 |

*Comparing downsampling schemas with including every data point

The aim of this project was to develop a method for assessing blood pressure stability or, put another way, a method for classifying a subject as one of hypotensive, hypotensive, unstable, or normal, based on the cumulative distribution of data points around a normative target blood pressure range and validate the method with respect to discrimination, responsiveness, and reliability properties. The outcome measures introduced herein, i.e., the area under the curve, the natural log of rate parameter of the fitted exponential curve with x-axis and y-axis intercepts, the fitting error, and the total deviation from the center of the target range (i.e., the target value, 115 mmHg), have been demonstrated as effective at quantifying blood pressure instability and deviation from clinically recommended values and that they are reliable, responsive, and discriminatory.

Traditionally, summary statistics, mainly mean and standard deviation or median, quartiles and extrema (minimum and maximum), have been used to measure blood pressure over time and response to treatments. The average provides the central tendency of the data over the recording period and can be highly discriminatory between two different recordings when the mean of the measurements changes. However, it fails to provide insights regarding how far the measurements are from a clinically accepted “normal” range and whether observed changes between two recordings indicates the data are trending closer or further from normal. Such insights are valuable to determine the clinical relevance of the change in distribution. Additionally, mean and standard deviation are highly sensitive to extreme occurrences—one aberrant blood pressure value can highly impact the accurate quantification of the blood pressure recorded over time as it skews the mean and inflates the standard deviation. Furthermore, in order to understand the distribution of the blood pressure over time, the median and both quartiles with extrema must be included. This makes it difficult to evaluate a group of individuals or use it as a study outcome measure.

For these reasons, it is more desirable to compare the blood pressure measurements to a clinically valid normative range. One option is to use the percentage of measurements falling within a pre-specified range. This method fails to account for values that inevitably fall outside the range, and given the variability inherent to cardiovascular function and the additional variability that occurs as a consequence of SCI, clinically relevant measurements that fall remarkably close to the boundaries will be ignored. This problem could be overcome by expanding the range, however, there is no consensus on how wide the range should be. The total deviation from a target line introduced herein addresses the boundary issue and provides an overall view of how far the measurements fall from a target. It is also demonstrated to be highly discriminatory between different recordings.

Despite this, total deviation alone does not comprehensively describe the distribution of data points with respect to the target. The proposed methodology based on cumulative distribution of blood pressure measurements (see FIGS. 2A-2G), overcomes this limitation. It accounts for values falling within and outside the range, can include extreme measures without being significantly influenced by them, and it provides a comprehensive model of the distribution of blood pressure measurements within a recording. In order to quantitatively describe the cumulative distribution curve (FIG. 2G), five of the six outcome measures are used: the area under the curve, which is directly calculated from the cumulative distribution curve; the rate parameter (log) of the fitted exponential curve and its x and y intercepts (which may be interpreted in terms of the sum of their normalized values

$$\frac{ln ( λ )}{4} + \frac{{Y}_{0}}{100} - {X}_{0} ) ,$$

as well as the fitting error. The analysis demonstrated all these proposed outcome measures are reliable, responsive, and discriminatory, and each measure describe a characteristic of blood pressure variability with respect to a normative range.

The sensitivity analysis demonstrated the commonly-used method of averaging blood pressure data points are detrimental to the blood pressure stability analysis: averaging essentially smooths the distribution and replaces actual recorded blood pressure values with artificial values calculated from smoothing. It is preferable to use all data points for the variability/stability analysis; if it is necessary to reduce the number of recordings, downsampling is preferred as it has significantly less impact on the stability measure outcomes than averaging. Results presented in Table 3 demonstrate total deviation is more sensitive to downsampling, followed by AUC; but outcome measures calculated from the exponential fitted curve

$$( \frac{ln ( λ )}{4} + \frac{{Y}_{0}}{100} - {X}_{0} and E )$$

are more robust and less affected by downsampling. Similarly, AUC is more sensitive to the chosen expansion rate but

$$\frac{ln ( λ )}{4} + \frac{{Y}_{0}}{100} - {X}_{0} and E$$

are not significantly affected by this factor.

The stability outcome measures presented herein are reliable, responsive, and discriminatory. They provide a solid ground for comprehensive and objective quantification of the effects of SCI on cardiovascular function, and can evaluate the effectiveness of clinical interventions that target blood pressure stability in individuals with chronic SCI. As most readily evident in FIGS. 4 and 7, the stability outcome measures can be used to evaluate and quantify the stability of blood pressure after SCI by comparing to people with normal blood pressure as well as showing improvement in cardiovascular regulation with the spinal cord stimulator in individuals with SCI. Sometimes treating hypotension in these individual can cause hypertension, which indicates that treatment was effective in changing the individual's blood pressure, but was not effective in regulating cardiovascular function into a desired target range. These outcome measures serve as guides for clinicians to determine whether treatment is resulting in quantifiable improvement in the cardiovascular function. Moreover when facing large datasets, the discriminatory properties of these outcome measures can be used to easily classify the individual subjects into various cardiovascular categories such as hypotensive, hypertensive, episodes between hyper-and hypotension (i.e., unstable) and normal blood pressure, as shown in FIGS. 3A-3C.

The foregoing detailed description is given primarily for clearness of understanding and no unnecessary limitations are to be understood therefrom for modifications can be made by those skilled in the art upon reading this disclosure and may be made without departing from the spirit of the invention.

1. A method for assessing blood pressure stability in a living subject, the method comprising the steps of:
measuring a blood pressure of a living subject over a period of time, said measuring including collecting a plurality of data points;
defining a target blood pressure range, said range including a blood pressure target value centered within the target blood pressure range;
calculating a total deviation of data points from the blood pressure target value;
defining a plurality of expanded blood pressure ranges;
plotting a cumulative distribution curve based on the data points and the plurality of expanded blood pressure ranges;
fitting an exponential cumulative function to the cumulative distribution curve;
determining a plurality of outcome measures, the plurality of outcome measures including the total deviation and at least one of an x-intercept of the exponential cumulative function, a y-intercept of the exponential cumulative function, λ, In(λ), and a fitting error calculated from said fitting; and
assessing blood pressure stability of the living subject based on the plurality of outcome measures.

2. The method of claim 1, wherein the measured blood pressure is a systolic blood pressure, wherein the blood pressure target range is a systolic blood pressure target range, wherein the blood pressure target value is a systolic blood pressure target value; and wherein the plurality of data points are a plurality of systolic blood pressure values collected over time.

3. The method of claim 1, wherein the target blood pressure range is 110 mmHg to 120 mmHg, and wherein the blood pressure target value is 115 mmHg.

4. The method of claim 1, further comprising, prior to calculating the total deviation of data points, omitting a first portion of data points above the target blood pressure range and omitting a second portion of data points below the target blood pressure range.

5. The method of claim 1, further comprising calculating an area under the curve (AUC) for the cumulative distribution curve, and wherein said plurality of outcome measures include the AUC.

6. The method of claim 1, wherein the exponential cumulative function is fitted to the cumulative distribution curve using the equation
$$Y = \{ \begin{matrix}( 1 - {e}^{- λ ( X - {X}_{0} )} ) + {Y}_{0} & X \geq 0 \\ 0 & X < 0\end{matrix} .$$

7. The method of claim 1, further comprising classifying the living subject as one of as one of hypotensive, hypertensive, unstable, or normal based on the assessment of blood pressure stability.

8. The method of claim 1, further comprising classifying the living subject as having stable or unstable blood pressure based on the assessment of blood pressure stability.

9. The method of claim 1, wherein the plurality of expanded blood pressure ranges vary by a predetermined expansion rate.

10. A method for classifying a living subject, the method comprising the steps of:
measuring a blood pressure of a living subject over a period of time, said measuring including collecting a plurality of data points;
defining a target blood pressure range, said range including a blood pressure target value centered within the target blood pressure range;
calculating a total deviation of data points from the blood pressure target value;
defining a plurality of expanding blood pressure ranges;
plotting a cumulative distribution curve based on the data points and the plurality of expanding blood pressure ranges;
calculating an area under the curve (AUC) for the cumulative distribution curve
fitting an exponential cumulative function to the cumulative distribution curve;
determining a plurality of outcome measures, the outcome measures including the total deviation, the AUC, and at least one of an x-intercept of the exponential cumulative function, a y-intercept of the exponential cumulative function, λ, In(λ), and a fitting error calculating from said fitting; and
classifying the subject as one of hypotensive, hypertensive, unstable, or normal based on the plurality of outcome measures.

11. The method of claim 10, wherein the exponential cumulative function is fitted to the cumulative distribution curve using the equation
$$Y = \{ \begin{matrix}( 1 - {e}^{- λ ( X - {X}_{0} )} ) + {Y}_{0} & X \geq 0 \\ 0 & X < 0\end{matrix} .$$

12. The method of claim 10, further comprising, prior to said calculating a total deviation of data points, downsampling the data points.

13. The method of claim 10, further comprising, prior to calculating the total deviation of data points, omitting a first portion of data points above the target blood pressure range and omitting a second portion of data points below the target blood pressure range.

14. The method of claim 13, wherein the first portion of data points is the highest data points above the target blood pressure range, and wherein the second portion of data points is the lowest data points below the target blood pressure range.

15. The method of claim 10, wherein the plurality of expanded blood pressure ranges vary by a predetermined expansion rate.

16. The method of claim 10, wherein the expansion rate is between 1 mmHg and 20 mmHg.

17. The method of claim 16, wherein the expansion rate is between 1 mmHg and 10 mmHg.
